(19) United States
(12) Patent Application Publication
(10) Pub. No.: US 2014/0099266 A1
(43) Pub. Date: Apr. 10, 2014

(54) MAGNETIC FLUID NANOSYSTEM

(71) Applicants: Janos BORBELY, Debrecen (HU); Istvan HAJDU, Tiszacsege (HU); Jozsef KOLLAR, Debrecen (HU); Magdolna BODNAR, Debrecen (HU); Zsuzsanna CSIKOS, Nyirbator (HU)

(72) Inventors: Janos BORBELY, Debrecen (HU); Istvan HAJDU, Tiszacsege (HU); Jozsef KOLLAR, Debrecen (HU); Magdolna BODNAR, Debrecen (HU); Zsuzsanna CSIKOS, Nyirbator (HU)

(73) Assignee: BBS NANOTECHNOLOGY LTD., Debrecen (HU)

(21) Appl. No.: 13/944,742

(22) Filed: Jul. 17, 2013

Related U.S. Application Data
(60) Provisional application No. 61/711,543, filed on Oct. 9, 2012.

(51) Int. Cl. A61K 49/18

(52) U.S. Cl. CPC A61K 49/1881
USPC 424/9.323; 424/9.32

(57) ABSTRACT

Targeting contrast agent for magnetic resonance imaging (MRI). In preferred embodiments, self-assembled polyelectrolytes coated superparamagnetic iron oxide contrast agent particles are provided, which are labeled with targeting moieties, afforded enhanced relaxivity, improved signal-to-noise and targeting ability. Accordingly, the invention relates to a stable targeting contrast nanosystem applicable for magnetic resonance imaging (MRI) having at least one nanoparticle polyelectrolyte polyanion; a targeting agent conjugated to the biopolymer; and a superparamagnetic ligand. In another embodiment the nanosystem according to the invention has at least two biocompatible and biodegradable nanoparticle polyelectrolyte biopolymer. Particularly, the superparamagnetic iron oxide particles are coated by a polyelectrolyte biopolymer and this system self-assembles with the other biopolymer to produce stable nanosystem for magnetic resonance imaging. Targeting moieties are conjugated to a biopolymer or to the self-assembled biopolymers to realize a targeted delivery of contrast agent. Methods for making these targeting MRI contrast agents are also provided.

This application takes the priority of U.S. Provisional Patent Application Ser. No. 61/711,543, filed on the 9^{th} of October, 2012, the entire content of which is incorporated herein by reference.

### BRIEF DESCRIPTION OF THE INVENTION

The present invention is directed to novel targeting contrast agent for magnetic resonance imaging (MRI). In preferred embodiments, self-assembled polyelectrolytes coated superparamagnetic iron oxide contrast agent particles are provided, which are labeled with targeting moieties, afforded enhanced relaxivity, improved signal-to-noise and targeting ability. Methods for making these targeting MRI contrast agents are also provided.

### FIELD OF THE INVENTION

The present invention relates to compositions useful as an MRI contrast agent and the preparation of said new MRI contrast agent, more specifically to self-assembled polyelectrolytes coated superparamagnetic iron oxide particles functionalized with targeting moiety, which are suitable for use as contrast agent in magnetic resonance imaging.

### BACKGROUND OF THE INVENTION

Magnetic resonance imaging (MRI) is one of the most important imaging techniques in the field of diagnostics. Advantages of MRI are that it is a noninvasive methodology and it provides excellent soft-tissue imaging contrast. MRI has developed rapidly and has become useful especially in the diagnosis and medication of neurological, cardiovascular and oncological diseases.

It is essentially important that images are hard-contrast, sensitive and have high stereoscopic resolution. Based upon physico-chemical differences, which can be exploited by varying light intensities (on the grey scale), pathological changes can be detected. The image contrast means a detectable difference between signal intensities, which induces optical stimulus to make diagnosis possible.

The resolution and the sensitivity of MRI can be enhanced by using intravascular contrast agents. Superparamagnetic and paramagnetic materials can be used as contrast agents, since they change the homogeneity of the magnetic field and alter the relaxation time of the tissue, where they reside, producing a hard-contrast.

Superparamagnetic agents may be magnetized to a substantially larger extent than paramagnetic agents can be, due to their higher magnetic moment, resulting in higher relaxivity. The superparamagnetic contrast agents can decrease the T2 (transversal) relaxation time and therefore can improve the contrast between the normal and diseased tissues.

Superparamagnetic agents such as superparamagnetic iron oxide nanoparticles (SPION) are excellent MR contrast agents. The contrast agent made from SPION is non-toxic, biocompatible, injectable and high-level accumulates in the targeted tissues or organs. However, SPION can easily aggregate and adsorb plasma protein due to their large surface. Other disadvantage of SPION is that rapidly eliminates by the mononuclear phagocytes systems and removes from the blood.

Ideally, a polymeric MRI contrast agent resides in the blood long enough and targets the studied (cancer) cells to produce hard-contrast in MRI and to allow completion of the imaging procedure; afterwards it should be degraded and excreted through the kidneys. A variety of studies have focused on the development of targeted nanoparticles, and dendrimers used for drug delivery. The tissue specificity and targeting property of contrast agents or combination of diagnosis and therapy open many new opportunities for cancer treatment.

Many recent attempts have been made to create contrast agents containing superparamagnetic iron oxide particles. These contrast agents give the most enhanced contrast in MR. The research works focus on the size of the iron oxide nanoparticles, because this property is the main factor for determination of their specific behavior.

Two main synthesis techniques can be discerned: (i) synthesis at high temperature and mechanical separation thereafter, and (ii) chemical synthesis in aqueous media. In chemical synthesis, surfactants are usually used for the stabilization of iron oxide nanoparticles.

Recently, both low molecular weight and carrier macromolecular moieties have attracted much interest because of their ability to improve MRI signals. However, the low-molecular weight contrast agents have serious shortcomings, such as short half-life in blood, rapidly diffuse out of the blood and excrete thought the kidney resulting in low image quality and lack of targeting specificity. In an effort to overcome these shortcomings, several macromolecular carriers have been developed, including proteins, polysaccharides, water-soluble fullerenes, carbon nanotubes, polymeric micelles, and other biocompatible natural and synthetic polymers, and polyelectrolyte complexes. Polyelectrolyte complexes offer many advantages, which include numerous reactive functional groups, the flexibility of the system and a lack of new covalent bond, which could modify the favorable biological properties of biopolymers.

The self-assembly of polyelectrolytes opens many new opportunities to develop a delivery system. The oppositely charged biopolymers can self-assemble by the attractive interaction between the functional groups. The electrostatic interactions between charged macromolecules can result stable self-assembled nanosystems, films or hydrogels. A variety of studies have focused on preparation and characterization of these polyelectrolyte complexes.

Chitosan (CH) is a renewable, basic linear biomaterial, containing β-[1→4]-linked 2-acetamido-2-deoxy-D-glucopyranose and 2-amino-2-deoxy-D-glucopyranose units. Currently, due to its special set of properties, which include low or non-toxicity, biocompatibility, biodegradability, low immunogenicity and antibacterial properties, chitosan has found wide application in a variety of areas, such as biomedicine, pharmaceuticals, metal chelation, food additives, and other industrial applications. A limiting factor in its application is its low solubility in aqueous media. However, chitosan can be solubilized by the protonation of its amino groups in acidic media, resulting in a cationic polysaccharide with high charge density.

Poly-γ-glutamic acid consists of repetitive glutamic acid units connected by amide linkages between α-amino and γ-carboxylic acid functional groups. The secondary structure of γ-PGA in aqueous solution has been described as an α-helix. Γ-PGA is different from other proteins, in that glutamate is polymerized via the non-peptide γ-amide linkages, and thus is synthesized in a ribosome-independent manner.

Γ-PGA is a water soluble, biodegradable, edible and nontoxic polyanion. Therefore, γ-PGA and its derivatives have been employed extensively in a variety of commercial applications such as cosmetics, food, medicine, and water treatment.

### BACKGROUND ART

Horak et al. (US 2009/0309597 A1) describe SPION synthesis and coating with saccharides and amino acids or poly(amino acid)s. The SPIO synthesis was performed in saccharide solutions and in situ precipitation was observed. Example 2 relates to SPIO particles coated with polyglutamic acid in a two steps synthesis. Efficiency of particles was investigated by MRI in vitro and in vivo.

Morteza et al. (US 20110223112) describe unsaturated polyester coated magnetic ultra-fine particles. The iron oxide particles were coated with poly(ethylene glycol fumarate) (PEGF). Characterization of these coated particles was performed and MTT test were used for the cytotoxicity assays.

Fahlvik et al. (U.S. Pat. No. 6,207,134 B1) relates to SPIO particles lightly coated with structural polysaccharides and synthetic polymers, especially polyaminoacids. Examples 17 and 18 describe magnetite particles coated with poly-L-glutamic acid in different reaction conditions. After sonication and centrifugation, the supernatant was filtered, and superparamagnetic particles were obtained.

Kyoungja Woo et al. (JMMM, 2009) describe folate targeted lipophilic iron oxide nanoparticles. This system accumulated significantly in KB cells, which overexpress folate receptors. They established that the cell specificity of the particles was correlation with the size of supraparamagnetic nanoparticles.

Kresse et al. (U.S. Pat. No. 6,576,221) describe nanoparticles consist of an iron-containing nanoparticles with double-coating: a primary coat, and a secondary coat (targeting polymer) and, optionally, of pharmaceutic adjuvants, pharmaceuticals, and/or adsorption mediators/enhancers. This nanosystem provides an opportunity to combine the diagnostics and therapy.

Wang et al. (20110085987) describe polyacrylic acid-bound iron oxide particles, which is targeted via folic acid adduct. The system is nontoxic and shows the superparamagnetic property. It can be performed as the chemotherapy agent and the contrast agent on magnetic resonance (MR) imaging.

Wu et al. (Polymer, 2006) describe the preparation of chitosan-poly(acrylic acid) polymer magnetic microspheres. First, a magnetic core was performed via self-assembly of positively charged chitosan and negatively charged iron oxide nanoparticles. Subsequently, acrylic acid monomers were polymerized on the magnetic cores to produce coated, stable, superparamagnetic microspheres.

### OTHER PUBLICATIONS

Hui Li Maa, Yu Feng Xub, Xian Rong Qi, Yoshie Maitani, Tsuneji Nagai: “Superparamagnetic iron oxide nanoparticles stabilized by alginate: Pharmacokinetics, tissue distribution, and applications in detecting liver cancers” International Journal of Pharmaceutics 354 (2008) 217-226.
J. Meng, J. Fan, G. Galian, R. T. Branca, P. L. Clasen, S. Ma, J. Zhou, C. Leuschner, C. S. S. R. Kumar, J. Hormes, T. Otiti, A. C. Beye, M. P. Harmer, C. J. Kiely, W. Warren, M. P. Haataja, W. O. Soboyejo: “LHRH-functionalized superparamagnetic iron oxide nanoparticles for breast cancer targeting and contrast enhancement in MRI” Materials Science and Engineering C 29 (2009) 1467-1479.
Chang-Moon Lee, hwan-Jeong Jeong, Se-Lim Kim, Eun-Mi Kim, Dong Wook Kim, Seok Tae Lim, Kyu Yoon Jang, Yong Yeon Jeong, Jae-Woon Nah and Myung-Hee Sohn: “SPION-loaded chitosan-linoleic acid nanoparticles to target hepatocytes” International Journal of Pharmaceutics 371 (2009) 163-169.
Jyun-Han Ke, Jia-Jyun Lin, James R. Carey, Jenn-Shing Chen, Chiao-Yun Chen, Li-Fang Wang: “A specific tumor-targeting magnetofluorescent nanoprobe for dual-modality molecular imaging” Biomaterials 31 (2010) 1707-1715.
Ramesh Kumar, B. Stephen Inbaraj, B. H. Chen: “Surface modification of superparamagnetic iron nanoparticles with calcium salt of poly(γ-glutamic acid) as coating material” Material Research Bulletin 45 (2010) 1603-1607.
Kyoungja Woo, Jihyung Moona, Kyu-SilChoi, Tae-Yeon Seong, Kwon-HaYoon: “Cellular uptake of folate-conjugated lipophilic superparamagnetic iron oxide nanoparticles” Journal of Magnetism and Magnetic Materials 321 (2009) 1610-1612.
Mohammad T. Islam, Istvan J. Majoros, James R. Baker Jr.: “HPLC analysis of PAMAM dendrimer based multifunctional devices” Journal of Chromatography B 822 (2005) 21-26.
Yan Wu, Jia Guo, Wuli Yang, Changchun Wang, Shoukuan Fu: “Preparation and characterization of chitosan-poly(acrylic acid) polymer magnetic microspheres” Polymer 47 (2006) 5287-5294.

### SUMMARY OF THE INVENTION

The present invention is directed to novel targeting contrast agent for magnetic resonance imaging. Accordingly, the invention relates to a stable targeting contrast nanosystem applicable for magnetic resonance imaging (MRI) comprising (i) at least one nanoparticle polyelectrolyte polyanion; (ii) a targeting agent conjugated to the biopolymer; (iii) a superparamagnetic ligand, preferably iron-oxide ligand, which is preferably nanoparticulate iron-oxide (SPION). In another embodiment the nanosystem according to the invention comprises (i) at least two, preferably water-soluble, biocompatible and biodegradable nanoparticle polyelectrolyte biopolymer. The composition may additionally contain a complexing agent and a formulating agent, though these are not necessarily included the composition. More particularly, the superparamagnetic iron oxide particles are coated by a polyelectrolyte biopolymer and this system self-assembles with the other biopolymer to produce stable nanosystem for magnetic resonance imaging. Targeting moieties are conjugated to a biopolymer or to the self-assembled biopolymers to realize a targeted delivery of contrast agent.

In some embodiments, these self-assembled particles internalize into the targeted tumor cells in consequence of the presence of targeting ligands. The internalized superparamagnetic contrast agents enhance relaxivity, improve the signal-to-noise and therefore conduce to early tumor diagnosis.

Furthermore, the present invention is directed to a method of making these, above mentioned targeting contrast agents, the method comprising the steps of a) synthesis of superparamagnetic iron oxide particles in the presence of at least one polyelectrolyte biopolymer; b) attaching the targeting molecules to the biopolymer coated iron-oxide particulate systems; and optionally c) mixing with the other biopolymer to give a stable, self-assembled, targeting MRI contrast agent. Furthermore, the invention relates to process for the preparation of a targeting contrast nanosystem according to the invention, comprising the steps of a) attaching the targeting molecules to the biopolymer, preferably PGA; then b) synthesis of superparamagnetic iron oxide particles in the presence of the material prepared in this step a) to give a stable, targeting MRI contrast agent.

### BRIEF DESCRIPTION OF THE DRAWINGS

FIG. 1. Schematic representation of formation of novel superparamagnetic, self-assembled, targeting nanoparticles

FIG. 2. Hydrodynamic size distribution of novel superparamagnetic, targeting nanoparticles

FIG. 3. Hydrodynamic size and size distribution of folated-poly-gamma-glutamic acid coated iron oxide (PFS).

FIG. 4. TEM micrograph of poly-gamma-glutamic acid coated superparamagnetic iron oxide.

FIG. 5. Time dependency of hydrodynamic size (a) and mobility (b) of SPION-loaded self-assembled nanoparticles.

FIG. 6. T₂-weighted MR images and T₂ relaxation time values of SPION-loaded self-assembled nanoparticles at different concentrations

FIG. 7. Confocal microscopic images of HeLa (A), HeDe (B), Jimt-1 (C), A2780 (D) and AD27820 (E) cells treated with SPION-loaded, self-assembled nanoparticles

FIG. 8. FACS analysis of untreated HeLa cells (control), and HeLa cells treated with SPION-loaded, self-assembled nanoparticles (a); and mean fluorescence intensities (FI) (b).

FIG. 9. T₂-weighted MR images of HeLa cells treated with folated-poly-gamma-glutamic acid coated iron oxide (PFS) (a) self assembled, SPION-loaded nanoparticles: PFS:CH=2:1 (b), PFS:CH=3:1 (c) and non treated control cells (d).

FIG. 10. MTT test of SPION-loaded, self-assembled nanoparticles using A2780 and MCF-7 cell lines

FIG. 11. MRI study on the uptake of SPION-loaded contrast agent in to HeLa cancer xenografts. T2 weighted MR images of control (a) and treated (b) SCID mice. Signal intensity values of the control tumor is 797+/−16 compared with the treated tumor 582+/−7

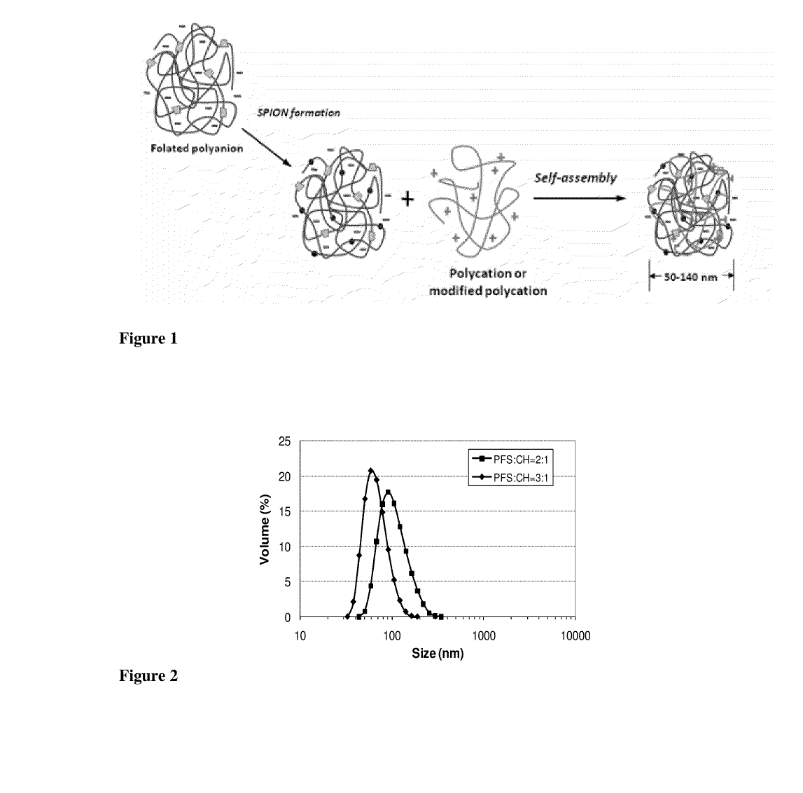

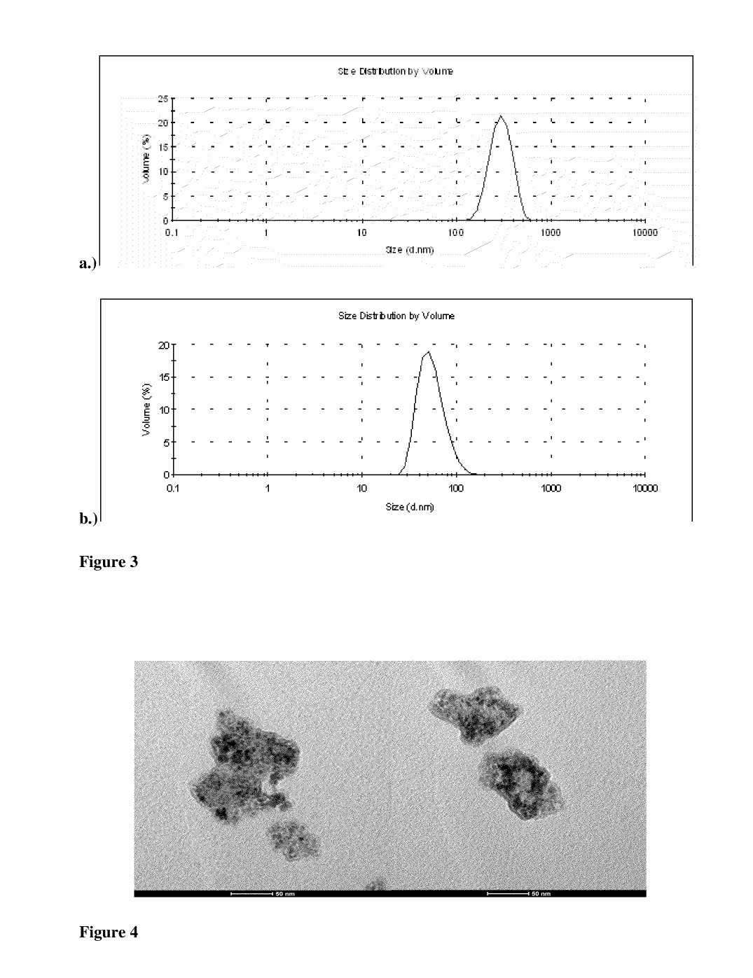

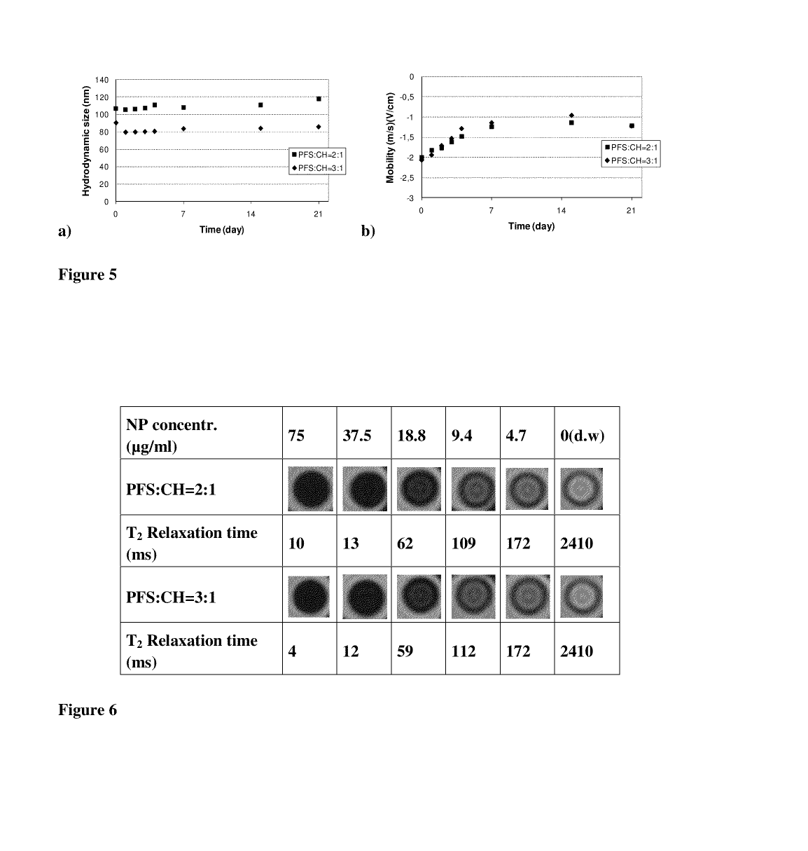

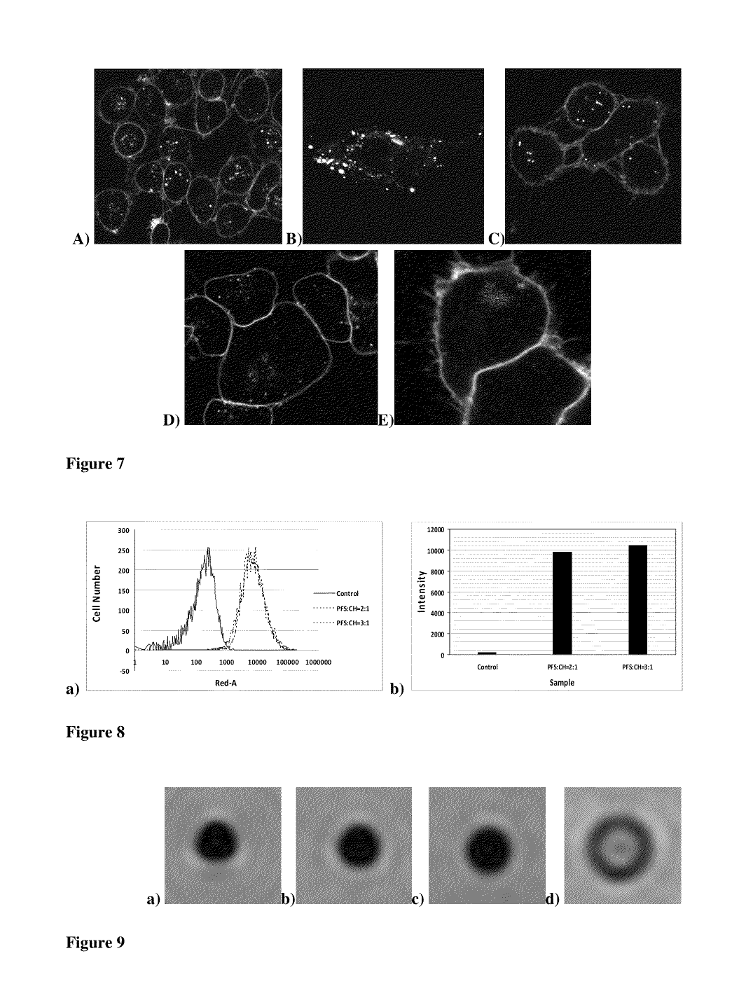

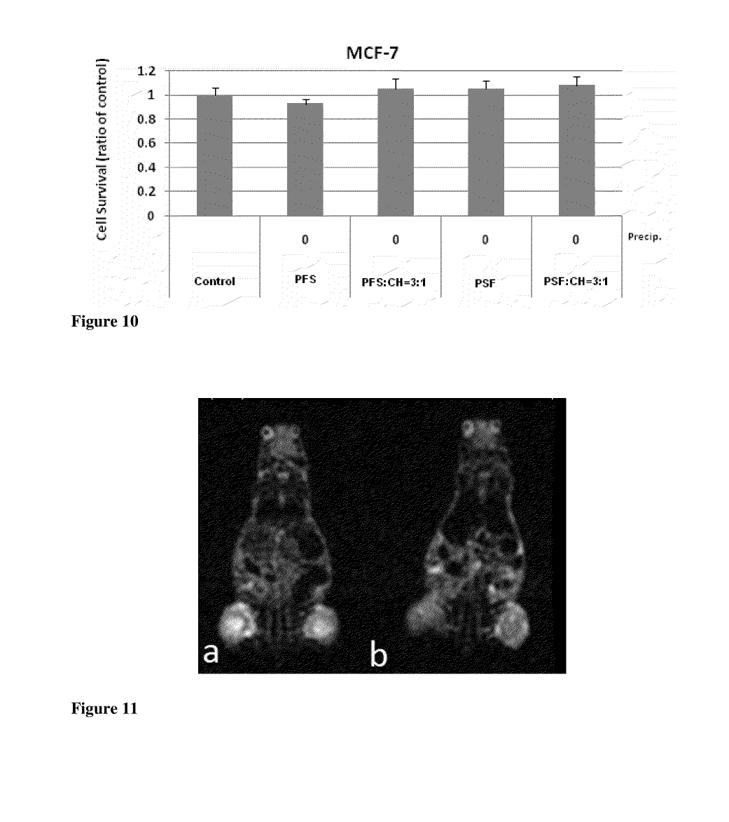

### DETAILED DESCRIPTION OF THE INVENTION

In one embodiment the present invention is directed to biocompatible, biodegradable nanoparticles, as superparamagnetic contrast agent formed by self-assembly via ion-ion interaction of oppositely charged functional groups of biopolymers. The nanoparticles of the present invention contain superparamagnetic ligand, preferably iron oxide nanoparticles, useful as MR contrast agent.

The present invention is directed to biocompatible, biodegradable nanoparticles, as contrast agent containing superparamagnetic iron oxide nanoparticles and targeting molecules.

In a preferred embodiment the superparamagnetic iron oxide nanoparticles (SPION) are synthesized in situ in the presence of the polyanion, and then the reaction with the polycation is performed.

In a preferred embodiment, the targeting agent is coupled to at least one of the polymers to achieve the specific accumulation of the nanoparticles in the targeted tumor cells. The present invention is directed to biocompatible, biodegradable nanoparticles as MRI contrast agent. Nanoparticles could be prepared by self-assembly of opposite charged polyelectrolytes to produce stable nanosystems.

In a preferred embodiment, the superparamagnetic nanoparticles contain at least three main components: (i) a polyanion containing SPIONs, (ii) a polycation and (iii) a targeting ligand coupled at least one the polyelectrolytes.

Based on the above, in its first aspect the present invention relates to a stable targeting contrast nanosystem applicable for magnetic resonance imaging (MRI) comprising (i) at least one, preferably water-soluble, biocompatible and biodegradable nanoparticle polyelectrolyte polyanion; (ii) a targeting agent conjugated to at least one polyelectrolyte biopolymer; (iii) a superparamagnetic ligand, preferably iron-oxide ligand, which is preferably nanoparticulate iron-oxide (SPION), which is preferably complexed to a polyelectrolyte biopolymer, and which is preferably homogenously dispersed; and (iv) optionally one or more formulating agent.

In its second aspect, the invention relates to a targeting contrast nanosystem applicable for magnetic resonance imaging (MRI), comprising (i) at least two, preferably water-soluble, biocompatible and biodegradable nanoparticle polyelectrolyte biopolymer; (ii) a targeting agent conjugated to at least one polyelectrolyte biopolymer; (iii) a superparamagnetic ligand, preferably iron-oxide ligand, which is preferably nanoparticulate iron-oxide (SPION), which is preferably complexed to a polyelectrolyte biopolymer, and which is preferably homogenously dispersed; and (iv) optionally one or more formulating agent.

In a preferred embodiment the targeting contrast according to the invention the superparamagnetic iron oxide particles are coated by a polyelectrolyte biopolymer. In another preferred embodiment of the present invention the superparamagnetic iron oxide particles are conjugated to the polyanion; and the targeting ligand is coupled to at least one of the polyelectrolytes.

In the nanosystem according to the invention at least one of the nanoparticle polyelectrolyte biopolymers is a polycation or a derivative thereof, preferably chitosan. In a preferred embodiment in the composition according to the invention the modified polycation is selected from the group of CH-EDTA, CH-DOTA, CH-DTPA. Preferably the complexing agent is selected from the group of diethylenetriaminepentaacetic acid (DTPA), 1,4,7,10-tetracyclododecane-N,-N′,N″,N′″-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethylether) N,N,N′,N′,-tetraacetic acid (EGTA), 1,4,8,11-tetraazacyclotradecane-N,N′,N″,N′″-tetraacetic acid (TETA), and 1,4,7-triazacyclononane-N,N′,N″-triacetic acid (NOTA), but is not limited to these materials.

The other polyelectrolyte biopolymer is a polyanion biopolymer or a derivative thereof, preferably selected from the group consisting of polyacrylic acid (PAA), poly-gamma-glutamic acid (PGA) hyaluronic acid (HA), and alginic acid (ALG), preferably poly-gamma-glutamic acid (PGA), said biopolymers being preferably self-assembled based on the ion-ion interactions between their functional groups.

The derivatives of biopolymers can be biopolymer-complexone products, or other grafted derivatives resulted in modifications of biopolymers with other molecules, e.g. PEG oligomers.

The formulating agent is selected from the group of glucose, physiological salt solution, phosphate buffered saline (PBS), sodium hydrogen carbonate.

The effects of glucose, sodium hydrogen carbonate, physiological saline solution, infusion base solutions and different buffers on the size, size distribution and stability of the nanoparticles were investigated. It was found that these solutions cause a decrease in the size distribution of the particles and accordingly, their stability will improve.

In its preferred embodiments the targeting contrast nanosystem according to the invention possesses one or more of the following features:

a) the superparamagnetic iron oxide nanoparticles are produced in situ in the polyanionic polymer; and/or
b) the polycation, preferably the chitosan, has a molecular weight from about 60 and 320 kDa, its pH ranges between 3.5 and 6, and its concentration ranges between 0.01 to 2 mg/ml; and/or
c) the polyanion, preferably the poly-gamma-glutamic acid (PGA) has a molecular weight between 10 kDa and 1.5 MDa, the pH of polyanion solution ranges between 7.5 and 10, and its concentration ranges between 0.01 to 2 mg/ml; and/or
d) the mass ratio of the polycation and the polyanion is between 1:20 and 20:1; and/or
e) the targeting agent is selected from the group of folic acid, LHRH, RGD and a monoclonal antibody, preferably folic acid; and/or
f) the nanosystem is in an aqueous solution.

In a preferred embodiment, in the targeting contrast nanosystem according to the invention the nanoparticles have a swollen hydrodynamic size between about 30 and 300 nm, preferably 50 and 140 nm, most preferably 80 and 110 nm, and size of dried SPIONs ranges between 1 and 15 nm, preferably 3 and 5 nm.

The nanoparticle compositions of present invention are prepared by mixing aqueous solution of biopolymers at given ratios and order of addition. In a preferred embodiment, aqueous solution of polycation and aqueous solution of polyanion are mixed to produce stable, self-assembled colloid systems. The polyelectrolytes have statistical distribution inside the nanoparticles to produce globular shape of the nanosystems. The order of addition influences the orientation of polyelectrolytes and affects the size and surface charge of the nanoparticles. For the in situ preparation of SPIO nanoparticles in the presence of a polyanion, Fe(III) ions are complexed to the biopolymer, and then SPION synthesis is started by the addition Fe(II) ions to the reaction mixture and raising the pH.

In a preferred embodiment, the SPION synthesis can be performed using several types of Fe(III) and Fe(II) ions, such as pl. FeCl₃×nH₂O (hydrate), Fe₂(SO₄)₃, Fe(NO₃)₃, Fe(III)-phosphate, FeCl₂×nH₂O, FeSO₄×nH₂O (hydrate), Fe(II)-fumarate, or Fe(II)-oxalate.

In a preferred embodiment, the concentration of polyanion was between 0.01-2.0 mg/ml, the ratio of Fe(III) and Fe(II) ions ranged between 5:1 and 1:5. The reaction takes place at elevated temperature ranging between 45 and 90° C. under N₂ atmosphere.

For the preparation of nanoparticles, the SPION-loaded polyanion and the polycation self-assemble and stable nanosystem is performed. The hydrodynamic size of swollen nanoparticles varies between 30 and 300 nm, preferably 50 and 140 nm, most preferably 80 and 110 nm. The size of dried SPIONs ranges between 1-15 nm, preferably 3-5 nm.

In a preferred embodiment, the mass ratio of polycation and polyanion can be between 1:20 and 20:1. The molecular weight of polyanion ranged between 10 kDa and 1.5 MDa, the pH of polyanion solution varied between 7.5 and 10, and its concentration could be 0.01-2 mg/ml. The molecular weight of polycation ranges between 60 and 320 kDa, the pH of polycation solution varied between 3.5 and 6, and its concentration could be 0.01-2 mg/ml.

Based on the above, the present invention relates to a process for the preparation of a targeting contrast nanosystem according to the invention, comprising the steps of

a) synthesis of superparamagnetic iron oxide particles in the presence of at least one polyelectrolyte biopolymer;
b) attaching the targeting molecules to the biopolymer coated iron-oxide particulate systems; and optionally
c) mixing with the other biopolymer
to give a stable, self-assembled, targeting MRI contrast agent; wherein the reaction preferably is run in an aqueous solution.

In another embodiment, the invention relates to process for the preparation of a targeting contrast nanosystem according to the invention, comprising the steps of

a) attaching the targeting molecules to the biopolymer, preferably PGA; then
b) synthesis of superparamagnetic iron oxide particles in the presence of the material prepared in step a)
to give a stable, targeting MRI contrast agent; wherein the reaction preferably is run in an aqueous solution.

In a preferred embodiment, in step a) of the above-mentioned processes

a) Fe(II) salt is added to the solution of the complex containing Fe(III) and a polyanion; and then
b) the pH and/or the temperature of the solution is increased to produce a complex of superparamagnetic iron oxide nanoparticles and a polyanionic polymer.

Preferably the process according to the invention possesses one or more of the following features:

a) as Fe(III) salt FeCl₃ or its hydrate, Fe₂(SO₄)₃, Fe(NO₃)₃, Fe(III)-phosphate is used; and/or
b) as Fe(II) salt FeCl₂ or its hydrate, FeSO₄ or its hydrate, Fe(II)-fumarate, or Fe(II)-oxalate is used; and/or
c) the concentration of the polyanion used ranges between 0.01-2.0 mg/ml; and/or
d) the ratio of the Fe(III) and Fe(II) ions used ranges between 5:1 and 1:5; and/or
e) the temperature used ranges between 45 and 90° C.; and/or
f) the reaction is run under N₂ atmosphere; and or
g) the concentration of the biopolymers ranges between 0.01 and 5 mg/ml.

The reaction conditions for examples speed of stirring, temperature, concentration of biopolymers, concentration and ratio of Fe(II) and Fe(III) ions greatly influence the size and size distribution of poly-gamma-glutamic acid coated iron oxide. The skilled person will be able to select the appropriate reaction conditions without undue experimentation.

The present invention provides tumor specific, SPION-loaded self-assembled nanoparticles. In a preferred embodiment, the targeting agent is preferably LHRH, RGD or folic acid, which facilitates the receptor mediated uptake of delivered nanoparticles.

In a preferred embodiment, the targeting ligand could be coupled to at least one of the polyelectrolytes. Depending on the binding place of targeting ligand, the size and surface charge of nanoparticles could be changed. For the coupling reaction of targeting molecules, the concentration of biopolymers could be ranged between 0.01 and 5 mg/ml.

In a preferred embodiment, the polyanion could be poly-gamma-glutamic acid, polyacrylic acid, or alginate, preferably poly-gamma-glutamic acid.

In a preferred embodiment, the polycation could be chitosan.

The present invention relates to SPION-loaded, self-assembled nanoparticles. The nanoparticles formation was performed via ion-ion interaction between functional groups of oppositely charged polyelectrolytes. The lack of covalent bonds between the biopolymers results that the biopolymers keep their favorable biological properties.

Efficiency of the nanoparticles was measured using several methods. Phantom MR investigation was performed to support that the nanoparticles can be change the homogeny magnetic field, change the relaxation time, and therefore could be effective MR contrast agent.

Internalization of nanoparticles into the targeted tumor cells was tested in vitro, using several tumor cell lines. Confocal microscopic and flow cytometric results supported that the nanoparticles internalized selectively into the targeted tumor cells.

The nanoparticles accumulate in the targeted tumor cells and transported superparamagnetion iron oxide nanoparticles, which statement was supports by MR investigation of targeted tumor cell suspensions treated with the superparamagnetic nanoparticles.

The biocompatibility of nanoparticles was controlled by MTT test using several tumor cell lines.

The present invention relates to tumor specific, SPION-loaded nanoparticles, as superparamagnetic MR contrast agents. Accordingly, the present invention relates to the use of the stable self-assembled targeting contrast nanosystem according to the invention in diagnosis. Preferably, the targeting contrast nanosystem is used in MR imaging; in cancer diagnosis. Accumulation of the nanoparticles and of transported SPION in the targeted cells reduced the relaxation time and changed the signal darkening of the MR images significantly. Results presented reveal that the superparamagnetic nanoparticles as targeted contrast agents exhibit excellent ability as T₂ contrast agent for MRI. These magnetic nanoparticles as targeting contrast agent could be a good candidate as T₂ contrast agent and open many exciting opportunities for targeted delivery of contrast agents to improve early tumor diagnosis.

### EXAMPLES

### Example 1

### Preparation of Folated Poly-Gamma-Glutamic Acid (PF)

Poly-gamma-glutamic acid (PGA) (m=130 mg) was dissolved in water (V=200 ml) and then adjusted to pH 5.8. Water soluble carbodiimide (m=26 mg) was added to the PGA solution and the reaction was stirred for 1 h at 4° C. and for another 1 h at room temperature. After the addition of folic acid (FA) (m=44 mg dissolved in 20 ml DMSO), the reaction mixture was stirred at 4° C. for 4 h then at room temperature for 20 h.

### Example 2

### Preparation of Folated Chitosan (CH-FA)

A solution of 1-(3-dimethylaminopropyl)-3-ethylcarbodiimide hydrochloride (CDI) and FA in anhydrous DMSO was prepared and stirred at room temperature until FA was well dissolved (1 h). Chitosan (CH) was dissolved in 0.1 M hydrochloric acid, to produce a solution with a concentration of 1 mg/ml, and then adjusted to pH 5.5 with 0.10 M sodium hydroxide solution. After the dropwise addition of CDI (m=5.1 mg in 1 ml distilled water) to the chitosan solution (V=20 ml), the reaction mixture was stirred for 10 min. Then folic acid (m=8.5 mg in dimethyl sulfoxide, V=1 ml) was added to the reaction mixture. The resulting mixture was stirred at room temperature in the dark for 24 h. It was brought to pH 9.0 by drop wise addition of diluted aqueous NaOH and was washed three times with aqueous NaOH, and once with distilled water. The polymer was isolated by lyophilization.

### Example 3

### Preparation of Chitosan-DTPA Conjugate (CH-DTPA)

Chitosan (m=15 mg) was solubilized in water (V=15 ml); its dissolution was facilitated by dropwise addition of 0.1 M HCl solution. After the dissolution, the pH of chitosan solution was adjusted to 5.0. After the dropwise addition of DTPA aqueous solution (m=11 mg, V=2 ml, pH=3.2), the reaction mixture was stirred at room temperature for 30 min, and at 4° C. for 15 min. after that, CDI (m=8 mg, V=2 ml distilled water) was added dropwise to the reaction mixture and stirred at 4° C. for 4 h, then at room temperature for 20 h. The chitosan-DTPA conjugate (CH-DTPA) was purified by dialysis.

### Example 4

### Preparation of Folated Chitosan-DTPA (CH-DTPA-FA)

A solution of CDI and FA in anhydrous DMSO was prepared and stirred at room temperature until FA was dissolved (1 h). The pH of chitosan-DTPA solution (c=1 mg/ml) was adjusted to pH 5.5 with 0.10 M sodium hydroxide solution. After the dropwise addition of CDI (m=5.1 mg in 1 ml distilled water) to the chitosan-DTPA solution (V=20 ml), the reaction mixture was stirred for 10 min. Then folic acid (m=8.5 mg in DMSO, V=1 ml) was added to the reaction mixture. The resulting mixture was stirred at room temperature in the dark for 24 h. It was brought to pH 9.0 by dropwise addition of diluted aqueous NaOH and was washed three times with aqueous NaOH, and once with distilled water. The chitosan-DTPA-FA was isolated by lyophilization.

### Example 5

### Preparation of Poly-Gamma-Glutamic Acid Coated Iron Oxide (PS)

PGA (m=10.5 mg) was dissolved in water (V=35 ml). The biopolymer solution was stirred under N₂ atmosphere for 30 min and then FeCl₃×6H₂O powder (m=18.2 mg) was added to the solution. The pH of the reaction mixture was raised to 8.0 and after that decreased to 6.0. After the stirring of the reaction mixture under N₂ atmosphere for 15 min, FeCl₂×4H₂O (m=16.7 mg) was added to it. The reaction mixture was stirred for 15 min under N₂ atmosphere, and then the pH was raised by addition of ammonium solution (V=4.5 ml, c=12.5 m/m %). Reaction temperature is 80° C., reaction time is 1 h.

### Example 6

### Preparation of Poly-Gamma-Glutamic Acid Coated Iron Oxide (PS)

FeCl₃×6H₂O (m=13.9 mg) was dissolved in water (V=27.8 ml) and then adjusted to pH 2.6. PGA (m=9 mg) was dissolved in water (V=30 ml) and then adjusted to pH 2.8.

The solutions were mixed, and the pH of the mixture was raised to 8.5, and after that it was decreased to 6.0. After the stirring of the reaction mixture under N₂ atmosphere for 30 min, FeCl₂×4H₂O (m=8.9 mg) was added to it. The reaction temperature was raised to 80° C., and then the pH was raised by addition of ammonium solution (V=3 ml, c=12.5 m/m %). Reaction time is 30 min

### Example 7

### Preparation of Folated Poly-Gamma-Glutamic Acid Coated Iron Oxide (PFS)

The pH of the folated PGA (prepared as described in Example 1) solution (c=0.3 mg/ml, V=30 ml) was adjusted to 2.8. After the dropwise addition of FeCl₃×6H₂O solution (c=0.5 mg/ml, V=13.9 ml), the pH of the reaction mixture was raised to 8.5 and after that it was reduced to 6.0. The reaction mixture was stirred for 30 min under N₂ atmosphere, and FeCl₂×4H₂O (m=8.9 mg) was added to the reaction mixture. Reaction temperature was raised to 80° C. and the pH was raised by addition of ammonium solution (V=3 ml, c=12.5 m/m %). Reaction time is 15 min Hydrodynamic size: 237 nm (FIG. 2a)

### Example 8

### Preparation of Folated Poly-Gamma-Glutamic Acid Coated Iron Oxide (PFS)

Folated PGA (prepared as described in Example 1) solution (c=0.3 mg/ml, V=35 ml) was stirred for 30 min under N₂ atmosphere, and FeCl₃×6H₂O powder (m=15.5 mg) was added to the solution. The pH of the reaction mixture was raised to 8.0 and after that reduced to 6.0. After the stirring of the reaction mixture under N₂ atmosphere for 15 min, FeCl₂×4H₂O (m=16.2 mg) was added to it. The reaction mixture was stirred for 15 min under N₂ atmosphere, and then the pH was raised by addition of ammonium solution (V=5 ml, c=12.5 m/m %). Reaction temperature is 80° C., reaction time is 30 min Hydrodynamic size: 60 nm (FIG. 2b)

### Example 9

### Reaction of Poly-Gamma-Glutamic Acid Coated Iron Oxide with Folic Acid (PSF)

The pH of the poly-gamma-glutamic acid coated iron oxide solution (V=30 ml, c=0.2 mg/ml) was adjusted to 5.8. The reaction mixture was stirred at 4° C. for 10 min, and after that cool CDI solution (m=1.3 mg, V=0.2 ml distilled water) was added dropwise to the reaction mixture. The reaction mixture was stirred at 4° C. for 1 h, then at room temperature for another 1 h. After the addition of folic acid (FA) (m=2.13 mg dissolved in 1 ml DMSO), the reaction mixture was stirred at 4° C. for 4 h then at room temperature for 20 h.

### Example 10

### Preparation of Self-Assembled, Folate-Targeted PGA/Chitosan Coated Iron Oxide Nanoparticles

### PFS:CH=3:1

1 ml of CH solution (c=0.3 mg/ml, pH=4) was added dropwise to 3 ml of PFS solution (c=0.3 mg/ml, pH=9)

### PFS:CH-EDTA=3:1

1 ml of CH-EDTA solution (c=0.3 mg/ml, pH=4) was added dropwise to 3 ml of PFS solution (c=0.3 mg/ml, pH=9)

### PFS:CH=2:1

1 ml of CH solution (c=0.1 mg/ml, pH=5) was added dropwise to 2 ml of PFS solution (c=0.1 mg/ml, pH=8.5)

### PS:CH-FA=3:1

1 ml of CH-FA solution (c=0.5 mg/ml, pH=5.5) was added dropwise to 3 ml of PFS solution (c=0.5 mg/ml, pH=9)

### PS:CH-EDTA-FA=3:1

1 ml of CH-EDTA-FA solution (c=0.5 mg/ml, pH=5.5) was added dropwise to 3 ml of PFS solution (c=0.5 mg/ml, pH=9)

### PS:PF:CH=1:1:1

1 ml of PS solution (c=0.3 mg/ml, pH=8) and 1 ml of PF solution (c=0.3 mg/ml, pH=8) were mixed, and 1 ml of CH solution (c=0.3 mg/ml, pH=5.5) was added to them dropwise.

### Example 11

### Characterization of Self-Assembled, SPION-Loaded Nanoparticles

The hydrodynamic size and size distribution of particles was measured using a dynamic light scattering (DLS) technique with a Zetasizer Nano ZS (Malvern Instruments Ltd., Grovewood, Worcestershire, UK). This system is equipped with a 4 mW helium/neon laser with a wavelength of 633 nm and measures the particle size with the noninvasive backscattering technology at a detection angle of 173°. Particle size measurements were performed using a particle-sizing cell in the automatic mode. The mean hydrodynamic diameter was calculated from the autocorrelation function of the intensity of light scattered from the particles. Electrokinetic mobility of the nanoparticles was measured in folded capillary cell (Malvern) with a Zetasizer Nano ZS apparatus.

### Example 12

### Cellular Uptake of Self-Assembled, SPION-Loaded Nanoparticles

Internalization and selectivity of nanoparticulates was investigated in cultured human cancer cells overexpressing folate receptors by using confocal microscopy and flow cytometry. The samples were imaged on an Olympus FluoView 1000 confocal microscope. Excitation was performed by using the 488 nm line of an Ar ion laser (detection: 500-550 nm) and the 543 nm line of a HeNe laser (detection: 560-610 nm) to image Alexa 488 and Alexa 546 respectively. Images were analyzed using Olympus FV10-ASW 1.5 software package. Flow cytometric analysis (BD FACSArray Bioanalyzer System) was carried out with a single-cell suspension, and only the live cells were gated based on forward and side scatter dot plots.

### Example 13

### MTT Assay of Self-Assembled, SPION-Loaded Nanoparticles

MTT assay of the SPION-loaded biopolymers and nanoparticles was performed using an UT-6100 Microplate Reader.

Approximately 10 000 cells/well were plated in 96-well plate. The cells were incubated at 37° C. for 24 h. After that the cells were treated with the SPION-loaded systems, and incubated at 37° C. for another 24 h. 20 μl MTT reagent was added to each well, and the plate was incubated for 4 h at 37° C. When purple precipitate was clearly visible under microscope, 200 μl DMSO was added to all wells, including control wells. The absorbance of the wells was measured at 492 nm.

### Example 15

### Effect of Glucose Solution on the Size and Polydispersity of Nanoparticles Through a Specific Example

Formulation of a nanoparticle (NP): mixing PFS (pH=9) and CH-EDTA (pH=4) in a ratio of 3:1, polymer concentration: 0.3 mg/ml

The nanoparticle is mixed with a 75% glucose solution in a ratio so that the final glucose concentration is 5%.

| | | Size | |
|---|---|---|---|
| | | of NP mixed | Polydispersity |
| Size of the | Polydispersity | with glucose | of NP mixed |
| original | of the | solution | with glucose |
| NP (nm) | original NP | (nm) | solution |
| 61 | 0.183 | 57 | 0.182 |

1. A stable targeting contrast nanosystem applicable for magnetic resonance imaging (MRI) comprising (i) at least one, preferably water-soluble, biocompatible and biodegradable nanoparticle polyelectrolyte polyanion; (ii) a targeting agent conjugated to at least one polyelectrolyte biopolymer; (iii) a superparamagnetic ligand, preferably iron-oxide ligand, which is preferably nanoparticulate iron-oxide (SPION), which is preferably complexed to a polyelectrolyte biopolymer, and which is preferably homogenously dispersed and (iv) optionally one or more formulating agent.

2. A stable targeting contrast nanosystem applicable for magnetic resonance imaging (MRI), comprising (i) at least two, preferably water-soluble, biocompatible and biodegradable nanoparticle polyelectrolyte biopolymer; (ii) a targeting agent conjugated to at least one polyelectrolyte biopolymer; (iii) a superparamagnetic ligand, preferably iron-oxide ligand, which is preferably nanoparticulate iron-oxide (SPION), which is preferably complexed to a polyelectrolyte biopolymer, and which is preferably homogenously dispersed; and (iv) optionally one or more formulating agent.

3. The targeting contrast nano system as claimed in claim 1, wherein the superparamagnetic iron oxide particles are coated by a polyelectrolyte biopolymer.

4. The targeting contrast nanosystem as claimed in claim 1, wherein the superparamagnetic iron oxide particles are conjugated to the polyanion; and the targeting ligand is coupled to at least one of the polyelectrolytes.

5. The targeting contrast nanosystem as claimed in claim 2, wherein at least one of the nanoparticle polyelectrolyte biopolymers is a polycation or a derivative thereof, preferably selected from the group of chitosan, CH-EDTA, CH-DOTA and CH-DTPA, wherein the chitosan preferably has a molecular weight from about 60 and 320 kDa, its pH ranges between 3.5 and 6, and its concentration ranges between 0.01 to 2 mg/ml.

6. The targeting contrast nanosystem as claimed in claim 1, wherein at least one of the nanoparticle polyelectrolyte biopolymers is a polyanion biopolymer or a derivative thereof, preferably selected from the group consisting of polyacrylic acid (PAA), poly-gamma-glutamic acid (PGA) hyaluronic acid (HA), and alginic acid (ALG), preferably poly-gamma-glutamic acid (PGA), said biopolymers being preferably self-assembled based on the ion-ion interactions between their functional groups.

7. The targeting contrast nanosystem as claimed in claim 1, wherein
a) the superparamegnetic iron oxide nanoparticles are produced in situ in the polyanionic polymer; and/or
b) the polyanion, preferably the poly-gamma-glutamic acid (PGA) has a molecular weight between 10 kDa and 1.5 MDa, the pH of polyanion solution ranges between 7.5 and 10, and its concentration ranges between 0.01 to 2 mg/ml; and/or
c) the mass ratio of the polycation and the polyanion is between 1:20 and 20:1; and/or
d) the targeting agent is selected from the group of folic acid, LHRH, RGD and a monoclonal antibody, preferably folic acid; and/or
e) the nanosystem is in an aqueous solution.

8. The targeting contrast nanosystem as claimed in claim 1, wherein the nanoparticles have a swollen hydrodynamic size between about 30 and 300 nm, preferably 50 and 140 nm, most preferably 80 and 110 nm, and size of dried SPIONs ranges between 1 and 15 nm, preferably 3 and 5 nm.

9. A process for the preparation of a targeting contrast nanosystem as claimed in claim 1, comprising the steps of
a) synthesis of superparamagnetic iron oxide particles in the presence of at least one polyelectrolyte biopolymer;
b) attaching the targeting molecules to the biopolymer coated iron-oxide particulate systems; and optionally
c) mixing with the other biopolymer
to give a stable, self-assembled, targeting MRI contrast agent; wherein the reaction preferably is run in an aqueous solution.

10. A process for the preparation of a targeting contrast nanosystem as claimed in claim 1, comprising the steps of
a) attaching the targeting molecules to the biopolymer, preferably PGA; then
b) synthesis of superparamagnetic iron oxide particles in the presence of the material prepared in step a)
to give a stable, targeting MRI contrast agent; wherein the reaction preferably is run in an aqueous solution.

11. The process as claimed in claim 9, wherein
a) Fe(II) salt is added to the solution of the complex containing Fe(III) and a polyanion; and then
b) the pH and/or the temperature of the solution is increased to produce a complex of superparamagnetic iron oxide nanoparticles and a polyanionic polymer.

12. The process as claimed in claim 9, wherein
a) as Fe(III) salt FeCl₃ or its hydrate, Fe₂(SO₄)₃, Fe(NO₃)₃, Fe(III)-phosphate is used; and/or
b) as Fe(II) salt FeCl₂ or its hydrate, FeSO₄ or its hydrate, Fe(II)-fumarate, or Fe(II)-oxalate is used; and/or
c) the concentration of the polyanion used ranges between 0.01-2.0 mg/ml; and/or
d) the ratio of the Fe(III) and Fe(II) ions used ranges between 5:1 and 1:5; and/or
e) the temperature used ranges between 45 and 90° C.; and/or
f) the reaction is run under N₂ atmosphere.

13. Use of the stable targeting contrast nanosystem as claimed in claim 1 in diagnosis.

14. The use as claimed in claim 13, wherein the targeting contrast nanosystem is used in MR imaging.

15. The use as claimed in claim 14, wherein the targeting contrast nanosystem is used in cancer diagnosis.

16. A method for improving the visibility of an internal body structure, said method comprising using the targeting contrast nanosystem of claim 1 in MR imaging.

17. The method of claim 16, wherein the internal body structure is a cancer tumor, wherein the method improves the early diagnosis thereof.
